(19) United States
(12) Patent Application Publication
(10) Pub. No.: US 2025/0226063 A1
(43) Pub. Date: Jul. 10, 2025

(54) INFORMATION PROCESSING APPARATUS AND MACHINE LEARNING METHOD

(71) Applicant: FUJITSU LIMITED, Kawasaki-shi (JP)

(72) Inventors: Sho TAKEMORI, Yokohama (JP); Yuhei UMEDA, Kawasaki (JP)

(73) Assignee: FUJITSU LIMITED, Kawasaki-shi (JP)

(21) Appl. No.: 19/088,790

(22) Filed: Mar. 24, 2025

Related U.S. Application Data
(63) Continuation of application No. PCT/JP2022/036413, filed on Sep. 29, 2022.

(51) Int. Cl. G16C 20/70; G06N 10/60

(52) U.S. Cl. CPC G16C 20/70; G06N 10/60

(57) ABSTRACT

A computer executes, among algorithms configured to obtain energy of a molecule through an iterative process and including a first algorithm and a second algorithm that uses quantum circuit data and is different from the first algorithm, the first algorithm based on molecular information specifying a molecule to be analyzed, to obtain a first iteration count of the first algorithm. The computer enters the first iteration count into a machine learning model trained with an iteration count of the first algorithm as an explanatory variable and an iteration count of the second algorithm as a response variable. The computer outputs an estimated value of a second iteration count of the second algorithm obtained from the machine learning model, for execution of the second algorithm based on the molecular information.

### CROSS-REFERENCE TO RELATED APPLICATION

This application is a continuation application of International Application PCT/JP2022/036413 filed on Sep. 29, 2022, which designated the U.S., the entire contents of which are incorporated herein by reference.

### FIELD

The embodiments discussed herein relate to an information processing apparatus and a machine learning method.

### BACKGROUND

Computers may perform molecular simulations to analyze the properties of molecules through numerical calculations. Molecular simulations may be used in industrial fields such as material development and pharmaceutical development. Molecular simulations include quantum chemical calculations that microscopically calculate the energy of a molecule on the basis of the electronic state of the molecule and the Schrödinger equation.

Quantum chemical calculation algorithms include algorithms using quantum circuit data, such as variational quantum eigensolver (VQE). It is also possible to use quantum computers to execute such algorithms using quantum circuit data. In addition, other quantum chemical calculation algorithms include configuration interaction (CI) methods and coupled cluster (CC) methods.

A typical algorithm performs an iterative process that iteratively calculates the energy of a molecule while changing its electron configuration. The algorithm may continue the iterative process until the calculated energy result converges. The algorithm may search for the electron configuration that minimizes the energy and output the minimum energy as the ground-state energy of the molecule.

For the configuration interaction methods, a quantum chemical computing device has been proposed that dynamically selects some molecular orbitals from a plurality of molecular orbitals of a molecule and calculates the energy of the molecule on the basis of electron configurations that are limited to the selected molecular orbitals.

See, for example, International Publication Pamphlet No. WO 2022/097298.

### SUMMARY

According to one aspect, there is provided a non-transitory computer-readable storage medium storing a computer program that causes a computer to perform a process including: executing, among algorithms configured to obtain energy of a molecule through an iterative process and including a first algorithm and a second algorithm that uses quantum circuit data and is different from the first algorithm, the first algorithm based on molecular information specifying a molecule to be analyzed, to obtain a first iteration count of the first algorithm; entering the first iteration count into a first machine learning model trained with an iteration count of the first algorithm as an explanatory variable and an iteration count of the second algorithm as a response variable; and outputting an estimated value of a second iteration count of the second algorithm obtained from the first machine learning model, for execution of the second algorithm based on the molecular information.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a view for describing an information processing apparatus according to a first embodiment;

FIG. 2 illustrates an example of the hardware configuration of an information processing apparatus according to a second embodiment;

FIG. 3 illustrates an example of comparison among different algorithms in terms of accuracy and execution time;

FIG. 4 illustrates an example of job scheduling;

FIG. 5 illustrates an example of input and output data of a time model and an iteration model;

FIG. 6 is a block diagram illustrating an example of the functions of the information processing apparatus;

FIG. 7 is a flowchart illustrating an example procedure for machine learning;

FIG. 8 is a flowchart illustrating an example procedure for execution time estimation; and

FIGS. 9A to 9C are graphs illustrating examples of execution time estimation accuracy.

### DESCRIPTION OF EMBODIMENTS

In some cases, a computer may estimate the iteration count of an algorithm that uses quantum circuit data, before executing the algorithm. For example, it is considered that the computer uses the estimated value of the iteration count to schedule a job that calculates molecular energy.

Note, however, that the iteration count of the algorithm may vary significantly depending on a molecule to be analyzed, and it is therefore not easy to achieve accurate estimation. For example, the iteration count needed until the calculation result of energy converges may differ greatly among molecules, and therefore it may be difficult to estimate the iteration count only from the quantum circuit data used by the algorithm.

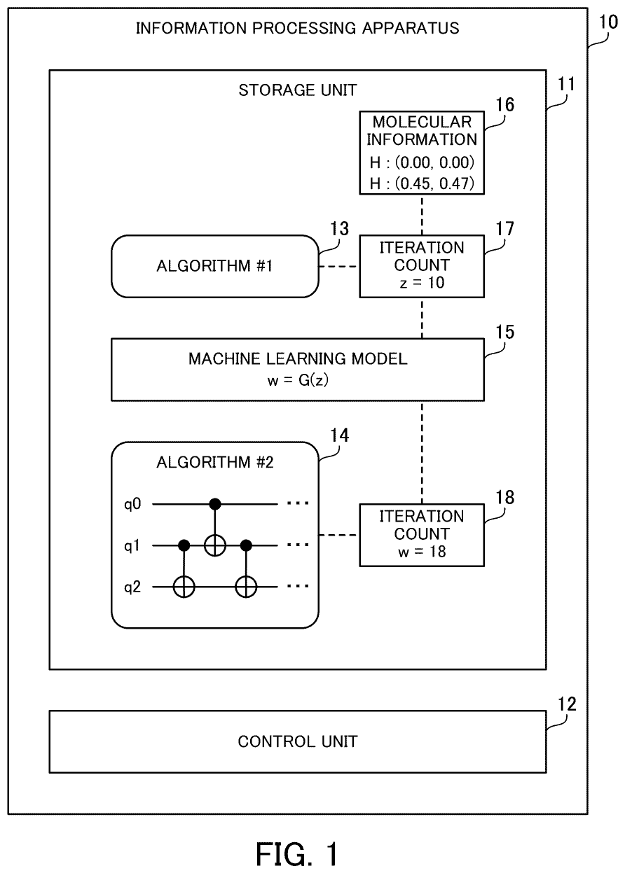

Hereinafter, embodiments will be described with reference to the accompanying drawings. A first embodiment will now be described. FIG. 1 is a view for describing an information processing apparatus according to the first embodiment. The information processing apparatus 10 estimates the iteration count of a quantum chemical calculation algorithm using a machine learning model.

The information processing apparatus 10 may train the machine learning model and may execute the algorithm for which the iteration count has been estimated. In addition, the information processing apparatus 10 may also schedule a quantum chemical calculation job on the basis of the estimated iteration count. The information processing apparatus 10 may be a client device or a server device. The information processing apparatus 10 may also be referred to as a computer, an estimation device, a machine learning device, a molecular simulation device, or a job scheduler.

The information processing apparatus 10 includes a storage unit 11 and a control unit 12. The storage unit 11 may be a volatile semiconductor memory, such as random access memory (RAM), or a non-volatile storage device, such as a hard disk drive (HDD) or flash memory.

The control unit 12 is, for example, a processor such as a central processing unit (CPU), a graphics processing unit (GPU), or a digital signal processor (DSP). Note that the control unit 12 may include an application specific integrated circuit (ASIC), a field programmable gate array (FPGA), or another electronic circuit. For example, the processor executes programs stored in a memory such as RAM (which may be the storage unit 11). The processor may be referred to as processor circuitry. A set of processors may be referred to as a multiprocessor or simply a “processor.” Different processors may perform different ones of a plurality of processes that will be described later.

The storage unit 11 stores a trained machine learning model 15. The machine learning model 15 may be a linear regression model, a nonlinear regression model, or a machine learning model other than regression models. The machine learning model 15 is trained with an iteration count of an algorithm 13 as an explanatory variable and an iteration count of an algorithm 14 as a response variable. Thus, the machine learning model 15 estimates the iteration count of the algorithm 14 from an iteration count of the algorithm 13. Another molecular feature value such as the distance between a plurality of atoms in a molecule may be included as the explanatory variable.

Each algorithm 13 and 14 is a quantum chemical calculation algorithm that calculates the energy of a molecule through an iterative process. For example, each algorithm 13 and 14 iteratively calculates the energy of a molecule while changing the electron configuration of the molecule, in order to obtain the minimum energy of the molecule as the ground-state energy of the molecule in the stable state. For example, the iterative process continues until the calculation result of energy satisfies a convergence condition. For example, the convergence condition may be that the difference between the most recent energy and its immediately previous energy is below a threshold.

The algorithm 14 is an algorithm that uses quantum circuit data. For example, the algorithm 14 is a variational quantum eigensolver (VQE). The algorithm 14 may be executed on a gate-based quantum computer or on a quantum simulator that simulates operations of a quantum computer on a Von Neumann-type classical computer. The algorithm 14 may be referred to as a quantum algorithm.

The quantum circuit data is a quantum computation model that defines gate operations on qubits. For example, the quantum circuit data defines an ansatz circuit that generates a quantum state using one or more qubits and a measurement circuit that applies a Hamiltonian to the quantum state to measure energy. The ansatz circuit and measurement circuit each include one or more quantum gates arranged in series. The quantum gates include controlled NOT (CNOT) gates, controlled Z (CZ) gates, phase shift gates, and Hadamard gates.

The quantum circuit data is generated from molecular information specifying a molecule to be analyzed. For example, the ansatz circuit is generated from basis functions representing molecular orbitals. The measurement circuit is generated based on Pauli matrices representing the Hamiltonian determined by the type of the molecule. The generation of the quantum circuit data may be considered as the preprocessing of the algorithm 14.

The algorithm 13 is different from the algorithm 14. For example, the algorithm 13 does not use quantum circuit data. The algorithm 13 is executed on a Von Neumann-type classical computer and may be referred to as a classical algorithm. It is preferable that the computational complexity and execution time of the algorithm 13 be sufficiently less than those of the algorithm 14. For example, the algorithm 13 may be a configuration interaction method or a coupled cluster method. Configuration interaction methods include configuration interaction singles and doubles (CISD). Coupled cluster methods include coupled cluster singles and doubles (CCSD) and coupled cluster singles and doubles (and triples) (CCSD(T)).

The control unit 12 obtains molecular information 16 specifying a molecule to be analyzed. For example, the molecular information 16 specifies the types of a plurality of atoms included in the molecule. In addition, for example, the molecular information 16 specifies the position coordinates of each atom included in the molecule. The molecular information 16 may be given by a user and stored in the storage unit 11. The control unit 12 estimates the iteration count for execution of the algorithm 14 based on the molecular information 16, before executing the algorithm 14.

First, the control unit 12 executes the algorithm 13 on the basis of the molecular information 16. Note that the control unit 12 may cause another information processing apparatus to execute the algorithm 13. Through the algorithm 13, the energy of the molecule specified by the molecular information 16 is calculated. At this time, the control unit 12 obtains an iteration count 17 of the algorithm 13 with regard to the molecular information 16. For example, the iteration count 17 indicates the number of iterations of the iterative process performed by the algorithm 13 until the energy calculated based on the molecular information 16 converges.

Then, the control unit 12 enters the iteration count 17 into the machine learning model 15. The machine learning model 15 calculates an iteration count 18 corresponding to the iteration count 17. The iteration count 18 is an estimated value of the iteration count for execution of the algorithm 14 based on the molecular information 16. For example, the iteration count 18 indicates the number of iterations of the iterative process that is performed by the algorithm 14 until the energy calculated based on the molecular information 16 converges. Although the iteration count 18 often does not match the iteration count 17, the iteration count 18 is positively correlated with the iteration count 17. Therefore, a higher iteration count 17 typically leads to a higher iteration count 18 being calculated.

Then, the control unit 12 outputs the estimated iteration count 18. The control unit 12 may store the estimated iteration count 18 in a non-volatile storage device, display it on a display device, or send it to another information processing apparatus.

In addition, the control unit 12 may estimate the execution time of the algorithm 14 with regard to the molecular information 16, using the iteration count 18. For example, the control unit 12 estimates the execution time by calculating the product of a unit execution time per iteration and the iteration count 18. The unit execution time may be a fixed value or may be estimated based on the scale of the quantum circuit data used by the algorithm 14. It may be possible to use a machine learning algorithm different from the machine learning model 15 to estimate this unit execution time.

The control unit 12 may output the estimated execution time. The control unit 12 may store the estimated execution time in a non-volatile storage device, display it on a display device, or send it to another information processing apparatus. Furthermore, the control unit 12 may control the execution of the algorithm 14 on the basis of the estimated execution time.

The control unit 12 may also schedule a job that calculates the energy of the molecule specified by the molecular information 16. For example, the control unit 12 may determine a hardware resource to allocate to the job on the basis of the estimated execution time. In addition, for example, the control unit 12 determines the scheduled start and end times of the job on the basis of the estimated execution time. Moreover, for example, on the basis of the estimated execution time, the control unit 12 may perform algorithm selection to determine whether to use the algorithm 14 or another algorithm.

As described above, the information processing apparatus 10 of the first embodiment executes the algorithm 13 on the basis of the molecular information 16 to obtain the iteration count 17 of the algorithm 13. The information processing apparatus 10 enters the iteration count 17 into the machine learning model 15, which has been trained with an iteration count of the algorithm 13 as an explanatory variable and an iteration count of the algorithm 14 as a response variable. The information processing apparatus 10 then outputs the estimated value of the iteration count 18 calculated by the machine learning model 15, for execution of the algorithm 14 based on the molecular information 16.

In the manner described above, the iteration count 18 of the algorithm 14 is estimated before the algorithm 14 is executed. This provides useful information that is usable for the execution control and job scheduling of the algorithm 14. In addition, the use of the iteration count 17 of the algorithm 13, which is correlated with the iteration count 18, improves the accuracy of estimating the iteration count 18. In particular, the estimation accuracy is improved compared to the case of estimating the iteration count 18 only from feature values representing features of the quantum circuit data.

Furthermore, an interatomic distance may be added in the explanatory variable of the machine learning model 15. The information processing apparatus 10 may enter the interatomic distance indicated by the molecular information 16, together with the iteration count 17 into the machine learning model 15. Even in the case where atoms in the molecule are of the same type, different iteration counts 18 are calculated depending on the interatomic distances. For example, a greater interatomic distance leads to a higher iteration count 18 being calculated. Thus, the accuracy of estimating the iteration count 18 is improved.

In addition, the information processing apparatus 10 may identify feature values of the quantum circuit data that is used for the execution of the algorithm 14 based on the molecular information 16. The information processing apparatus 10 may enter the identified feature values into another machine learning model that has been trained with feature values of quantum circuit data as an explanatory variable and a unit execution time per iteration of an iterative process as a response variable. The information processing apparatus 10 may then output the estimated value of the unit execution time of the algorithm 14 calculated by the other machine learning model. This provides useful information that is usable for the execution control and job scheduling of the algorithm 14.

The information processing apparatus 10 may also estimate the execution time of the algorithm 14 on the basis of the estimated value of the iteration count 18 and the estimated value of the unit execution time. This provides useful information that is usable for the execution control and job scheduling of the algorithm 14, and improves the accuracy of estimating the execution time of the algorithm 14. In particular, the estimation accuracy is improved compared to the case of estimating the execution time only from feature values of the quantum circuit data.

In addition, the information processing apparatus 10 may schedule a job that calculates molecular energy, on the basis of the estimated execution time. As a result, the job scheduling is performed based on the highly accurate estimated execution time, which improves the utilization efficiency of hardware resources, such as server devices and processors, and also achieves an earlier job completion time.

Furthermore, the algorithm 13 may be a configuration interaction method or a coupled cluster method, and the algorithm 14 may be a variational quantum eigensolver. This allows the iteration count 18 to be estimated quickly in a significantly shorter time than the execution time of the algorithm 14. In addition, since the iteration count 17 is positively correlated with the iteration count 18, the accuracy of estimating the iteration count 18 is improved.

Moreover, the information processing apparatus 10 may train the machine learning model 15 as follows. For example, the information processing apparatus 10 executes the algorithms 13 and 14 on the basis of molecular information specifying a sample molecule, to obtain both the iteration count of the algorithm 13 and the iteration count of the algorithm 14. Using training data including thus obtained iteration counts, the information processing apparatus 10 trains the machine learning model 15 that has the iteration count of the algorithm 13 as an explanatory variable and the iteration count of the algorithm 14 as a response variable.

For example, the information processing apparatus 10 performs the fitting of a regression model used as the machine learning model 15 to optimize the parameter values included in the machine learning model 15. This improves the accuracy of estimating the iteration count of the algorithm 14.

A second embodiment will now be described. An information processing apparatus 100 of the second embodiment schedules a quantum chemical calculation job that calculates molecular energy. For the scheduling, the information processing apparatus 100 estimates the execution time of a VQE before executing the VQE.

A quantum chemical calculation algorithm may be executed by the information processing apparatus 100 or by another information processing apparatus. In addition, a machine learning model that is used to estimate the execution time may be trained by the information processing apparatus 100 or by another information processing apparatus. The information processing apparatus 100 may be a client device or a server device. The information processing apparatus 100 may also be referred to as a computer, an estimation device, a machine learning device, a molecular simulation device, or a job scheduler. The information processing apparatus 100 corresponds to the information processing apparatus 10 of the first embodiment.

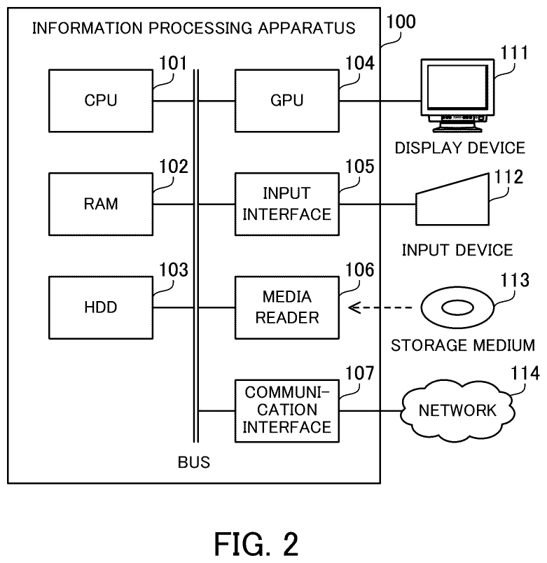

FIG. 2 illustrates an example of the hardware configuration of the information processing apparatus according to the second embodiment. The information processing apparatus 100 includes a CPU 101, a RAM 102, an HDD 103, a GPU 104, an input interface 105, a media reader 106, and a communication interface 107, which are connected to a bus. The CPU 101 corresponds to the control unit 12 of the first embodiment. The RAM 102 or HDD 103 corresponds to the storage unit 11 of the first embodiment.

The CPU 101 is a processor that executes program commands. The CPU 101 loads programs and data from the HDD 103 to the RAM 102 and executes the programs. The information processing apparatus 100 may be provided with a plurality of processors.

The RAM 102 is a volatile semiconductor memory that temporarily stores programs being executed by the CPU 101 and data being used by the CPU 101 in processing. The information processing apparatus 100 may be provided with a different type of volatile memory than RAM.

The HDD 103 is a non-volatile storage device that stores software programs such as an operating system (OS), middleware, and application software, and data. The information processing apparatus 100 may be provided with another type of non-volatile storage device such as flash memory or solid state drive (SSD).

The GPU 104 performs image processing in collaboration with the CPU 101 and outputs images to a display device 111 connected to the information processing apparatus 100. Examples of the display device 111 include a cathode ray tube (CRT) display, a liquid crystal display, an organic electro-luminescence (EL) display, and a projector. Another type of output device such as a printer may be connected to the information processing apparatus 100.

The GPU 104 may be used as a general-purpose computing on graphics processing unit (GPGPU). The GPU 104 is able to execute programs in accordance with instructions from the CPU 101. The information processing apparatus 100 may be provided with a volatile semiconductor memory other than the RAM 102 as a GPU memory.

The input interface 105 receives an input signal from an input device 112 connected to the information processing apparatus 100. Examples of the input device 112 include a mouse, a touch panel, and a keyboard. A plurality of input devices may be connected to the information processing apparatus 100.

The media reader 106 is a reading device that reads programs and data from a storage medium 113. Examples of the storage medium 113 include a magnetic disk, an optical disc, and a semiconductor memory. Magnetic disks include flexible disks (FDs) and HDDs. Optical discs include compact discs (CDs) and digital versatile discs (DVDs). The media reader 106 copies the programs and data from the storage medium 113 to another storage medium such as the RAM 102 or the HDD 103. The read programs may be executed by the CPU 101.

The storage medium 113 may be a portable storage medium. The storage medium 113 may be used to distribute the programs and data. The storage medium 113 and HDD 103 may be referred to as computer-readable storage media.

The communication interface 107 communicates with other information processing apparatuses over a network 114. The communication interface 107 may be a wired communication interface that is connected to a wired communication device such as a switch or a router, or may be a wireless communication interface that is connected to a wireless communication device such as a base station or an access point.

The following describes algorithms for quantum chemical calculation. The quantum chemical calculation is one type of molecular simulation and analyzes molecular structures and intermolecular interactions from electronic states. The quantum chemical calculation is sometimes used to assist material development and pharmaceutical development. The quantum chemical calculation is microscopic molecular simulation, and achieves high analytical accuracy but has high computational load.

A quantum chemical calculation solves the Schrödinger equation, HΨ=EΨ, where H denotes a Hamiltonian, Ψ denotes a wave function, and E denotes energy. The Hamiltonian H depends on a target molecular structure. The wave function Ψ describes an electronic eigenstate, and the energy E is an eigenenergy corresponding to Ψ. The quantum chemical calculation calculates the ground-state energy when the molecular structure is stable. However, it is difficult to directly solve the Schrödinger equation.

To address this, in the quantum chemical calculation, the wave function Ψ is expressed as basis functions. Each basis function is represented as a linear combination of known functions, and each term in the basis function corresponds to a molecular orbital. The molecular orbital is a location where any one of the electrons in the molecule may enter. The quantum chemical calculation receives the specification of molecular information indicating the position coordinates of the plurality of atoms in the molecule, a solution-finding algorithm, and basis functions, and calculates the ground-state energy on the basis of the specified information.

The quantum chemical calculation may generate a potential energy curve. This potential energy curve represents potential energies corresponding to different interatomic distances. A potential energy represents the energy that the molecule would have when each atom is assumed to be at rest. The horizontal axis of the potential energy curve represents the distance between two specified atoms, whereas the vertical axis thereof represents the ground-state energy.

The unit of distance is, for example, angstrom (Å). The unit of energy is, for example, hartree. Energy is calculated for each of a plurality of discrete distances within a certain range. These distances may be equally spaced. For example, the energy may be calculated at 0.1 Å intervals from 0.5 Å to 3.0 Å. The potential energy curve is generated by plotting the calculated energies and connecting the plotted points with a line. The minimum point on the potential energy curve may represent the most stable state of the molecule. The maximum point on the potential energy curve may represent a transition state of the molecule.

Quantum chemical calculation jobs may include jobs that calculate the energies of different types of molecules. In addition, the quantum chemical calculation jobs may include jobs that calculate the energies of molecules that are of the same type but have different distance settings. The quantum chemical calculation jobs may also include jobs requested by different users.

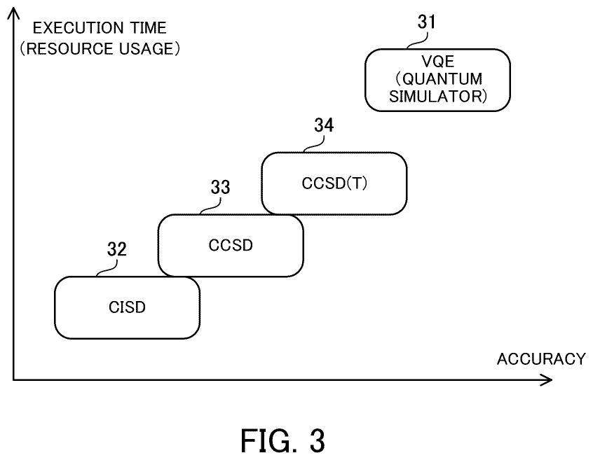

FIG. 3 illustrates an example of comparison among different algorithms in terms of accuracy and execution time. Quantum chemical calculation algorithms include classical algorithms designed to run on von Neumann-type classical computers and quantum algorithms designed to run on gate-based quantum computers. Note, however, that the quantum algorithms may be executed using software that simulates operations of a quantum computer and a classical computer. In the second embodiment, it is assumed that the quantum algorithms are executed using such a simulator.

Examples of the quantum chemical calculation algorithms include algorithms 31, 32, 33, and 34, as illustrated in FIG. 3. The algorithm 31 is a VQE, which is classified as a quantum algorithm. The algorithm 32 is a CISD, which is classified as a configuration interaction method. The algorithm 33 is a CCSD, and the algorithm 34 is a CCSD(T). Both the CCSD and the CCSD(T) are classified as coupled cluster methods. The CISD, CCSD, and CCSD(T) belong to classical algorithms.

The VQE generates a quantum circuit that generates a quantum state using a plurality of qubits on the basis of specified basis functions. This quantum circuit may be called an ansatz circuit. In addition, the VQE generates a quantum circuit that measures energy from the quantum state on the basis of the Hamiltonian corresponding to specified molecular information. This quantum circuit may be called a measurement circuit. Each quantum circuit is a quantum computation model represented as a combination of quantum gates. The generation of the quantum circuits may be referred to as the preprocessing of the VQE.

In the case of using a quantum computer, a quantum circuit is implemented with physical qubits. In the case of using a quantum simulator, pseudo-qubit data is stored in a memory, and pseudo-quantum gate operations are implemented using classical programs.

The VQE uses the ansatz circuit to generate a quantum state and then uses the measurement circuit to measure energy. Each measurement has been affected by noise and fluctuations. Therefore, for the same electron configuration, the VQE generates a quantum state and measures energy a plurality of times, and then calculates the average of these measurements as an expected value of energy. The VQE adjusts the parameter values used for generating the quantum state so as to make the expected value of energy smaller. An adjustment in the parameters is equivalent to a change in the electron configuration. By iteratively performing the above process, the VQE searches for the ground-state energy. For example, the VQE iteratively performs the above process until the expected value of energy converges.

In the case of using a quantum simulator, each time a single qubit is added to a classical computer, its memory usage and computational amount get double. The VQE exhibits higher solution accuracy than the classical algorithms. However, the VQE has a significantly longer execution time than the classical algorithms. The VQE may have an execution time that is 1,000 times longer than that of a classical algorithm.

The CISD obtains an approximate solution to the Schrödinger equation with the Born-Oppenheimer approximation on the basis of specified molecular information and basis functions. The CISD takes the effects of single- and double-electron excitations on energy into account while disregarding the effects of triple- and higher order-electron excitations. The CISD iteratively calculates changing the energy while electron configuration, in order to search for the minimum energy. The CISD performs the iterative process until the calculated energy converges. For example, the CISD compares the solution of the most recent iteration with that of its immediately previous iteration, and completes the iterative process when the difference between these two solutions falls below a threshold.

In general, the CISD exhibits lower solution accuracy than the VQE, CCSD, and CCSD(T). However, the CISD has a shorter execution time than the VQE, CCSD, and CCSD(T). The CISD has a computational complexity of an order that is the sixth power of the number of electrons.

The CCSD is an algorithm based on the Hartree-Fock molecular orbital method and approximates the wave function of a multi-electron molecule using an exponential cluster operator that takes electron correlation into account. The CCSD obtains an approximate solution to the Schrödinger equation with the approximated wave function on the basis of specified molecular information and basis functions. The CCSD takes the effects of single- and double-electron excitations on energy into account while disregarding triple- and higher order-electron excitations. As with the CISD, the CCSD iteratively calculates energy while changing the electron configuration, in order to search for the minimum energy. The CCSD performs the iterative process until the calculated energy converges.

In general, the CCSD exhibits lower solution accuracy than the VQE and CCSD(T) but higher solution accuracy than the CISD. In addition, the CCSD has a shorter execution time than the VQE and CCSD(T) but a longer execution time than the CISD. The CCSD has a computational complexity of an order that is the sixth power of the number of electrons.

The CCSD(T), as well the CCSD, is classified as a coupled-cluster method. The CCSD(T), however, takes different electron excitations from the CCSD into account. Specifically, the CCSD(T) precisely calculates the effects of single- and double-electron excitations on energy and obtains the effect of triple-electron excitation through perturbations, as an electron state. On the other hand, the CCSD(T) disregards the effects of quadruple- and higher order-electron excitations.

In general, the CCSD(T) exhibits lower solution accuracy than the VQE but higher solution accuracy than the CISD and CCSD. In addition, the CCSD(T) has a shorter execution time than the VQE but a longer execution time than the CISD and CCSD. The CCSD(T) has a computational complexity of an order that is the seventh power of the number of electrons.

A molecule to be simulated is sometimes referred to as a “system.” An execution time may be positively correlated with and proportional to resource usage. The hardware resources used in quantum chemical calculations may include the command execution time of a processor and the storage region of a memory. The classical algorithms are able to calculate molecular energy without using quantum circuits.

The following describes job scheduling. For the job scheduling, the information processing apparatus 100 may allocate resources to two or more VQE jobs requested by the same user or different users. These two or more VQE jobs may be executed by different computing nodes, or at different times by the same computing node.

In this case, the information processing apparatus 100 estimates the execution time for each of the two or more VQE jobs. The information processing apparatus 100 may determine a start time for each VQE job on the basis of the estimated execution times, and may notify the user(s) of the scheduled completion times. In addition, the information processing apparatus 100 may allocate a plurality of computing nodes to the VQE jobs on the basis of the estimated execution times so as to balance the load among the computing nodes. The information processing apparatus 100 may also guarantee that the VQE jobs are completed by user-specified deadlines, on the basis of the estimated execution times.

As part of the job scheduling, the information processing apparatus 100 may automatically select an algorithm on the basis of an estimated execution time. The algorithm selection determines whether to use the VQE or a classical algorithm for calculating the energy of a user-specified molecule. If sufficient resources are available on a computing node, the information processing apparatus 100 may select the VQE, which achieves higher accuracy. If sufficient resources are not available on a computing node, on the other hand, the information processing apparatus 100 may select the classical algorithm, which has a shorter execution time. The information processing apparatus 100 may select an algorithm on the basis of a user-specified deadline and the estimated execution time.

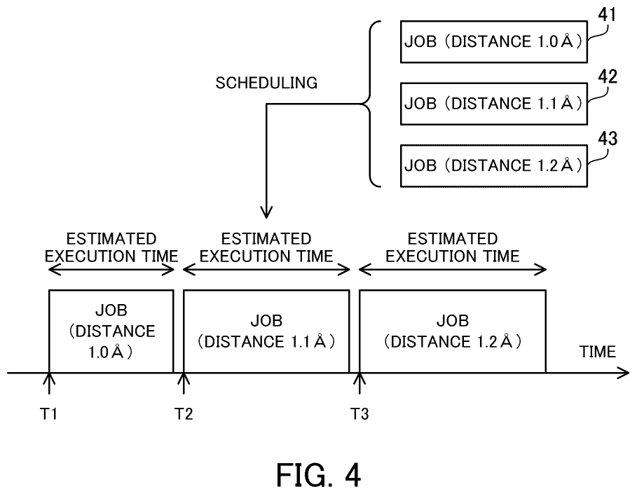

FIG. 4 illustrates FIG. an example of job scheduling. In this example, the information processing apparatus 100 schedules jobs 41, 42, and 43. The jobs 41, 42, and 43 are requested by the same user and are intended to calculate the energies of molecules that are of the same molecular type but have different interatomic distances. The jobs 41, 42, and 43 are executed using the VQE. For simple description, it is assumed that the jobs 41, 42, and 43 are executed on a single computing node. The computing node that executes the jobs 41, 42, and 43 may be the information processing apparatus 100 itself.

The information processing apparatus 100 generates a schedule for causing the computing node to sequentially execute the jobs 41, 42, and 43. To this end, the information processing apparatus 100 estimates the execution time for each job 41, 42, and 43 on the basis of specified molecular information. The information processing apparatus 100 determines the start and end times for each job 41, 42, and 43 on the basis of the estimated execution times.

For example, the information processing apparatus 100 sets the start time of the job 41 to time T1. In addition, the information processing apparatus 100 sets the start time of the job 42 to time T2, which is later than the time T1. For example, the time T2 is the time obtained by adding the estimated execution time of the job 41 to the time T1. Similarly, the information processing apparatus 100 sets the start time of the job 43 to time T3, which is later than the time T2. For example, the time T3 is the time obtained by adding the estimated execution time of the job 42 to the time T2.

If a job is not completed by the start time of the following job, the information processing apparatus 100 may forcibly terminate that job. For this reason, each estimated execution time for the jobs 41, 42, and 43 may be calculated to exceed its expected value with taking such a risk into account.

The following describes the estimation of execution time for the VQE. The information processing apparatus 100 estimates the execution time of the VQE using machine learning models trained in advance. The machine learning models may be referred to as estimators. The machine learning models of the second embodiment are Gaussian process regression models generated by a Gaussian process. In addition, the information processing apparatus 100 generates training data using sample molecular information and trains the machine learning models using the generated training data.

The machine learning models include a time model that estimates the execution time per iteration of the VQE and an iteration model that estimates the iteration count of the VQE. The execution time per iteration corresponds to the time needed to calculate an for a single electron expected value of energy configuration. The iteration count corresponds to the number of attempts in each of which the electron configuration is changed to search for the ground-state energy. The estimated value of the execution time of the VQE is calculated as the product of the unit execution time estimated by the time model and the iteration count estimated by the iteration model.

As described above, in the second embodiment, the estimation of the execution time for the iterative process is divided into the estimation of execution time per iteration and the estimation of iteration count. This is because it is difficult to accurately estimate the iteration count only from the scale of the quantum circuit used by the VQE. In addition, even in the case where molecules have the same interatomic distance, the iteration counts for the molecules needed until convergence may be different depending on their molecular types.

Moreover, the actual iteration count may fluctuate due to randomness, and there is therefore an upside risk of the actual iteration count exceeding the expected value. In addition, due to a small amount of training data, uncertainty may arise in the estimation result obtained by the iteration model. To address these, the information processing apparatus 100 may use an iteration model that outputs an iteration count exceeding the expected value, with taking at least one of the randomness and uncertainty into account. The following describes an example of the machine learning models using mathematical formulae.

First, the time model that estimates an execution time per iteration will be described. The explanatory variable of the time model is a three-dimensional vector x defined by Formula (1), where q denotes the number of qubits, d denotes the depth of an ansatz circuit, and l denotes the number of terms in a Hamiltonian. The depth of the ansatz circuit refers to the number of stages of the quantum gates arranged in series. The number of terms in the Hamiltonian refers to the number of terms obtained by decomposing the Hamiltonian into a sum of Pauli matrices.

$$x = ( q , d , ℓ ) \qquad ( 1 )$$

The time model, which calculates the expected value of the execution time per iteration, is defined as Formula (2), for example. In Formula (2), y is a response variable representing the execution time per iteration, and n denotes the number of records in training data. Training data to be used for training the time model includes n records, each containing a value of the explanatory variable and a value of the response variable as a pair, represented as (x₁, y₁), . . . , (xₙ, yₙ).

$$y = ( {y}_{1} , … , {y}_{n} ) {( {K}_{n} + λ {I}_{n} )}^{- 1} {k}_{n} ( x ) \qquad ( 2 )$$

Denote k as the kernel of a Gaussian process. The kernel k is a function that defines the similarity between vectors. Examples of the kernel k include a radial basis function (RBF) kernel and a Matern kernel. In Formula (2), Kₙ is an n×n square matrix generated from values of the explanatory variable in the training data. The element in the i-th row and j-th column of the matrix Kₙ is k(xᵢ, xⱼ). The matrix Kₙ represents the similarity between two values of the explanatory variable in the training data. Iₙ is an n×n identity matrix. kₙ(x) is a column vector whose i-th row element is k(xᵢ, x). kₙ(x) indicates the similarity between a certain vector x and each of n values of the explanatory variable included in the training data. λ is a constant greater than zero.

Considering the risk of an actual execution time per iteration deviating from an expected value, the information processing apparatus 100 is able to use a time model that takes robustness against that risk into account. First, as seen in Formula (3), a conditional value at risk (CVaR) is defined for the execution time per iteration. In Formula (3), α is a constant greater than zero and less than or equal to one. ψ_{ν}(y) and U are defined as in Formula (4).

$${CVaR}_{n , α} ( x ; {y}_{1} , … , {y}_{n} ) = {max}_{v ϵ U} \{ v - \frac{1}{α} ( {ψ}_{v} ( {y}_{1} ) , … , {ψ}_{v} ( {y}_{n} ) ) {( {K}_{n} + λ {I}_{n} )}^{- 1} {k}_{n} ( x ) \} \qquad ( 3 )$$

$${ψ}_{v} ( y ) = max ( v - y , 0 ) , U = \{ {y}_{1} , … , {y}_{n} \} \qquad ( 4 )$$

For example, the time model that takes the robustness into account is defined as Formula (5) using CVaR of Formula (3). The estimated value calculated by Formula (5) reflects the upside risk of the execution time per iteration and is therefore expected to be greater than the expected value calculated by Formula (2). Denoting ρ as a distribution of the vector x and denoting F as a cumulative distribution function corresponding to the distribution ρ, Formula (5) provides an estimated value of Formula (6).

$$- {CVaR}_{n , 1 - α} ( x ; {y}_{1} , … , - {y}_{n} ) \qquad ( 5 )$$

$${E}_{y ∼ ρ ( x )} [ y \vert y \geq {F}^{- 1} ( α ) ] \qquad ( 6 )$$

In addition, further considering uncertainty in the estimation of the time model due to insufficient training data, the information processing apparatus 100 is able to use a time model that takes both the robustness and the uncertainty into account. First, as seen in Formula (7), σₙ(x) is defined for the execution time per iteration. In Formula (7), k^{T}ₙ(x) is the transpose matrix of kₙ(x).

$${σ}_{n} ( x ) = \sqrt{k ( x , x ) - {k}_{n}^{T} ( x ) {K}_{n}^{- 1} {k}_{n} ( x )} \qquad ( 7 )$$

The time model that takes both the robustness and the uncertainty into account is defined as Formula (8) using, for example, βₙ(x) of Formula (7). In Formula (8), β is a positive constant. The estimated value calculated by Formula (8) reflects an additional upside risk of the execution time per iteration and is therefore greater than the estimated value calculated by Formula (5).

$$- {CVaR}_{n , 1 - α} ( x ; {y}_{1} , … , {y}_{n} ) + {βσ}_{n} ( x ) \qquad ( 8 )$$

Next, the iteration model that estimates an iteration count will be described. The basic structure of the iteration model is the same as that of the time model. Note, however, that the explanatory variable and response variable in the iteration model have different definitions from those in the time model. The explanatory variable of the iteration model is a two-dimensional vector z as defined by Formula (9), where m denotes an iteration count of a classical algorithm and s denotes an interatomic distance.

One classical algorithm is selected in advance. For example, one is selected from the CISD, CCSD, and CCSD(T). In the following description, it is assumed that the CCSD is selected. Note that, in a broad sense, the term “CCSD” may be interpreted as including both the narrow definitions of the CCSD and CCSD(T). In the case where a molecule contains three or more atoms, the interatomic distance s is calculated as the average of the distances between two atoms. The distance may be, for example, the Euclidean distance.

$$z = ( m , s ) \qquad ( 9 )$$

The iteration model that estimates an iteration count is defined as, for example, Formula (10), where w is a response variable representing the iteration count of the VQE. Training data to be used for training the iteration model includes n records, each containing a value of the explanatory variable and a value of the response variable as a pair, represented as (z₁, w₁), . . . , (zₙ, wₙ).

$$w = ( {w}_{1} , … , {w}_{n} ) {( {L}_{n} + λ {I}_{n} )}^{- 1} {ℓ}_{n} ( z ) \qquad ( 10 )$$

In Formula (10), denote l as the kernel of a Gaussian process. Lₙ is an n×n square matrix generated from values of the explanatory variable included in the training data. The element in the i-th row and j-th column of the matrix Lₙ is l(zᵢ, zⱼ). lₙ(z) is a column vector whose i-th row element is l(zᵢ, z). λ is a constant greater than zero.

As with the time model, considering the risk of an actual iteration count deviating from an expected value, the information processing apparatus 100 is able to use an iteration model that takes robustness against that risk into account. For example, the iteration model that takes the robustness into account is defined as Formula (11) using CVaR of Formula (3). Here, in Formulae (3) and (4), x is replaced with z, y is replaced with w, Kₙ is replaced with Lₙ, and kₙ is replaced with lₙ.

$$- {CVaR}_{n , 1 - α} ( z ; - {w}_{1} , … , - {w}_{n} ) \qquad ( 11 )$$

In addition, further considering uncertainty in the estimation of the iteration model due to insufficient training data, the information processing apparatus 100 is able to use an iteration model that takes both the robustness and the uncertainty into account. For example, the iteration model that takes both the robustness and the uncertainty into account is defined as Formula (12) using Formula (7). Here, in Formula (7), x is replaced with z, Kₙ is replaced with Lₙ, and kₙ is replaced with lₙ.

$$- {CVaR}_{n , 1 - α} ( z ; - {w}_{1} , … , - {w}_{n} ) + {βσ}_{n} ( z ) \qquad ( 12 )$$

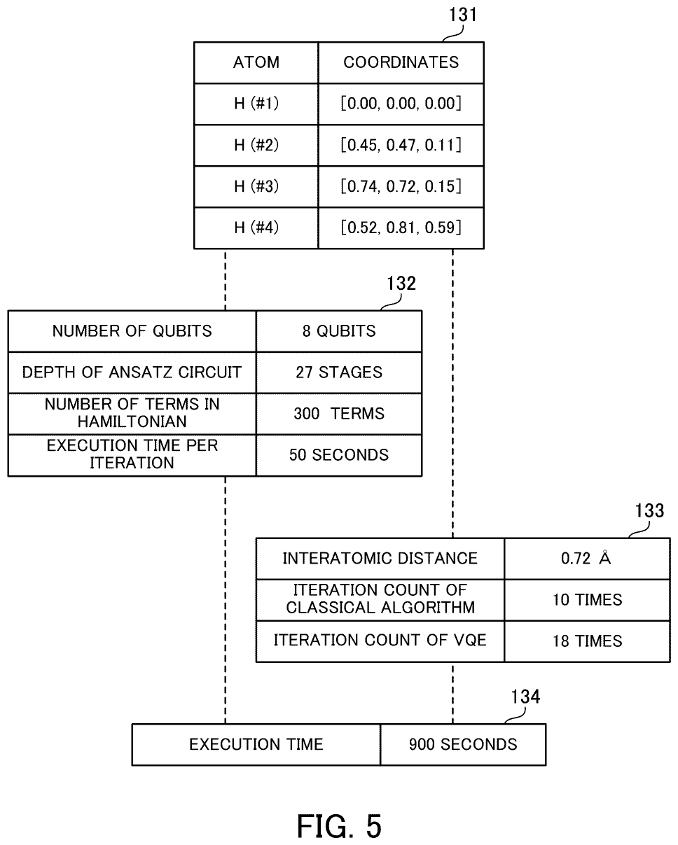

FIG. 5 illustrates an example of input and output data of a time model and an iteration model. The information processing apparatus 100 obtains data 131 on a molecule to be analyzed. The data 131 indicates the type and coordinates of each atom in the molecule. At the time of machine learning, the information processing apparatus 100 obtains n sets of data that is like the data 131 as sample data.

The information processing apparatus 100 generates data 132 from the data 131. The data 132 includes the number of qubits, the depth of an ansatz circuit, the number of terms in a Hamiltonian, and an execution time per iteration. The number of qubits, the depth of the ansatz circuit, and the number of terms in the Hamiltonian are input data to the time model and are calculated from the data 131 through the preprocessing of the VQE. The execution time per iteration is output data from the time model.

At the time of the machine learning, the information processing apparatus 100 generates n sets of data that is like the data 132 as training data to be used for training the time model. In this case, the execution time per iteration corresponds to teacher data and is measured by executing the VQE.

In addition, the information processing apparatus 100 generates data 133 from the data 131. The data 133 includes an interatomic distance, an iteration count of a classical algorithm, and an iteration count of the VQE. The interatomic distance and the iteration count of the classical algorithm are input data to the iteration model. The interatomic distance is calculated from the coordinates of each atom indicated in the data 131. The iteration count of the classical algorithm is measured by executing the classical algorithm on the basis of the data 131. The iteration count of the VQE is output data from the iteration model.

At the time of the machine learning, the information processing apparatus 100 generates n sets of data that is like the data 133 as training data to be used for training the iteration model. In this case, the iteration count of the VQE corresponds to teacher data and is measured by executing the VQE. The information processing apparatus 100 is able to measure both the execution time per iteration and the iteration count of the VQE together.

The information processing apparatus 100 generates data 134 from the data 132 and 133. The data 134 includes an estimated value of the execution time of the VQE. The execution time is calculated as the product of the execution time per iteration included in the data 132 and the iteration count of the VQE included in the data 133. One or both of the execution time per iteration output from the time model and the iteration count of the VQE output from the iteration model may each represent an expected value, an estimated value obtained by taking robustness into account, or an estimated value obtained by taking both robustness and uncertainty into account. The user may specify an estimated value type according to the intended use of the estimated execution time.

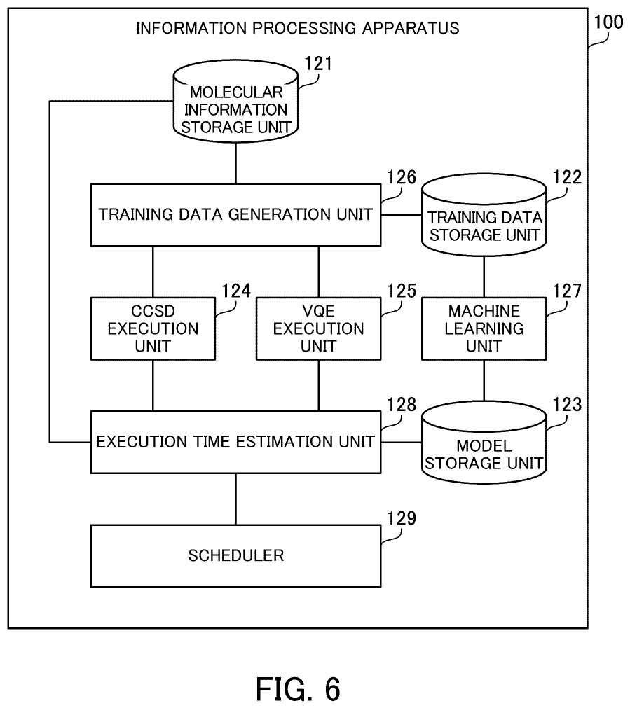

The following describes the functions and processing procedures of the information processing apparatus 100. FIG. 6 is a block diagram illustrating an example of the functions of the information processing apparatus. The information processing apparatus 100 includes a molecular information storage unit 121, a training data storage unit 122, and a model storage unit 123. These storage units are implemented by using, for example, the RAM 102 or HDD 103. In addition, the information processing apparatus 100 includes a CCSD execution unit 124, a VQE execution unit 125, a training data generation unit 126, a machine learning unit 127, an execution time estimation unit 128, and a scheduler 129. These processing units are implemented by using, for example, the CPU 101 or GPU 104 and programs.

The molecular information storage unit 121 stores sample molecular information to be used in machine learning. The molecular information storage unit 121 also stores molecular information specifying a molecule to be analyzed. This molecular information is generated by a user. The above-described data 131 is stored in the molecular information storage unit 121.

The training data storage unit 122 stores training data to be used for training a time model that estimates an execution time per iteration. The training data storage unit 122 also stores training data to be used for training an iteration model that estimates the iteration count of the VQE. These training data is generated based on the sample molecular information stored in the molecular information storage unit 121. The above-described data 132 and 133 are stored in the training data storage unit 122. The model storage unit 123 stores the time model and the iteration model as trained machine learning models.

The CCSD execution unit 124 executes the CCSD, which a is representative classical algorithm. Alternatively, the information processing apparatus 100 may execute the CISD or CCSD(T). The CCSD execution unit 124 executes the CCSD on the basis of the sample molecular information and basis functions specified by the training data generation unit 126, to perform an iterative process until the energy converges. The CCSD execution unit 124 measures the iteration count and outputs it to the training data generation unit 126.

In addition, the CCSD execution unit 124 executes the CCSD on the basis of the molecular information on the molecule to be analyzed and basis functions specified by the execution time estimation unit 128, to perform an iterative process until the energy converges. The CCSD execution unit 124 measures the iteration count and outputs it to the execution time estimation unit 128.

The VQE execution unit 125 executes the VQE using a quantum simulator. Alternatively, the VQE execution unit 125 may use a quantum computer. The VQE execution unit 125 performs the preprocessing on the basis of the sample molecular information and basis functions specified by the training data generation unit 126, to generate a quantum circuit appropriate for the sample molecule. The VQE execution unit 125 then executes the VQE on the basis of the generated quantum circuit, to perform an iterative process until the energy converges. The VQE execution unit 125 measures the execution time per iteration and the iteration count and outputs them together with the generated quantum circuit to the training data generation unit 126.

In addition, the VQE execution unit 125 performs the preprocessing on the basis of the molecular information on the molecule to be analyzed and basis functions specified by the execution time estimation unit 128, to generate a quantum circuit appropriate for the molecule to be analyzed. The VQE execution unit 125 outputs the generated quantum circuit to the execution time estimation unit 128. The VQE execution unit 125 also executes the VQE on the basis of the generated quantum circuit according to a schedule specified by the scheduler 129.

The training data generation unit 126 generates the training data to be used for training the time model and the training data to be used for training the iteration model and stores them in the training data storage unit 122. For each piece of sample molecular information, the training data generation unit 126 acquires a quantum circuit from the VQE execution unit 125 to extract the feature values of the quantum circuit, and acquires an execution time per iteration from the VQE execution unit 125. Thereby, the training data for the time model is generated.

In addition, for each piece of sample molecular information, the training data generation unit 126 calculates an interatomic distance on the basis of the molecular information, acquires an iteration count from the CCSD execution unit 124, and acquires an iteration count from the VQE execution unit 125. Thereby, the training data for the iteration model is generated.

The machine learning unit 127 trains the time model and the iteration model through a Gaussian process using the training data stored in the training data storage unit 122, and stores the trained time model and iteration model in the model storage unit 123. The machine learning unit 127 may train a machine learning model that outputs an expected value, a machine learning model that takes robustness into account, or a machine learning model that takes both robustness and uncertainty in account. The user may specify to the machine learning unit 127 whether to take robustness and uncertainty into account.

The execution time estimation unit 128 uses machine learning models stored in the model storage unit 123 to estimate an execution time for execution of the VQE based on molecular information on a molecule to be analyzed. The execution time estimation unit 128 outputs the estimated execution time to the scheduler 129. More specifically, for the molecular information on the molecule to be analyzed, the execution time estimation unit 128 acquires a quantum circuit from the VQE execution unit 125 to extract the feature values of the quantum circuit. The execution time estimation unit 128 enters the feature values of the quantum circuit into the time model to estimate the execution time per iteration.

In addition, for the molecular information on the molecule to be analyzed, the execution time estimation unit 128 calculates an interatomic distance on the basis of the molecular information and acquires an iteration count from the CCSD execution unit 124. The execution time estimation unit 128 enters the interatomic distance and the iteration count of the CCSD into the iteration model to estimate the iteration count of the VQE. The execution time estimation unit 128 then estimates the execution time of the VQE by multiplying the estimated execution time per iteration by the estimated iteration count of the VQE.

The scheduler 129 schedules a job that calculates the energy of the molecule to be analyzed on the basis of the estimated execution time received from the execution time estimation unit 128. For example, the scheduler 129 selects based on the estimated execution time whether to use the VQE or a classical algorithm (e.g., CCSD(T)) to calculate the energy of the molecule to be analyzed. In addition, the scheduler 129 schedules a plurality of VQE jobs (e.g., the start time for each VQE job) on the basis of estimated execution times.

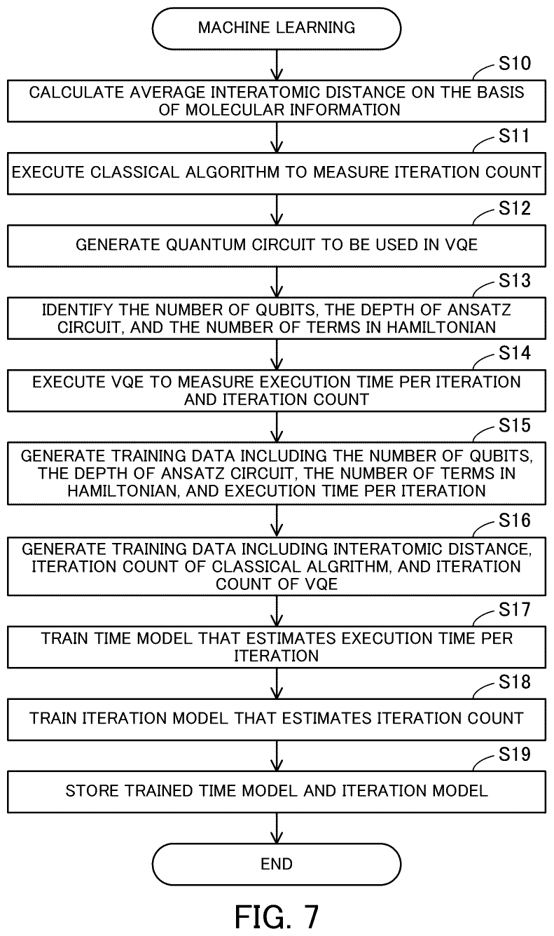

FIG. 7 is a flowchart illustrating an example procedure for machine learning. (S10) The training data generation unit 126 acquires sample molecular information. The training data generation unit 126 calculates the average interatomic distance on the basis of the molecular information.

(S11) The CCSD execution unit 124 executes a classical algorithm (e.g., CCSD) on the basis of the molecular information and basis functions to measure the iteration count of the classical algorithm.

(S12) The VQE execution unit 125 performs the preprocessing of the VQE on the basis of the molecular information and basis functions to generate a quantum circuit to be used in the VQE.

(S13) The training data generation unit 126 identifies the number of qubits, the depth of an ansatz circuit, and the number of terms in a Hamiltonian on the basis of the preprocessing result obtained at step S12.

(S14) The VQE execution unit 125 executes the VQE on the basis of the preprocessing result obtained at step S12 to measure the execution time per iteration of the VQE and the iteration count of the VQE.

(S15) The training data generation unit 126 generates training data including the number of qubits, the depth of the ansatz circuit, and the number of terms in the Hamiltonian, identified at step S13, and the execution time per iteration measured at step S14.

(S16) The training data generation unit 126 generates training data including the interatomic distance calculated at step S10, the iteration count of the classical algorithm measured at step S11, and the iteration count of the VQE measured at step S14.

(S17) The machine learning unit 127 trains a time model that estimates an execution time per iteration, using the training data generated at step S15.

(S18) The machine learning unit 127 trains an iteration model that estimates the iteration count of the VQE, using the training data generated at step S16.

(S19) The machine learning unit 127 stores the time model trained at step S17 and the iteration model trained at step S18 in a non-volatile storage device. In addition, the machine learning unit 127 may display the trained time model and iteration model on the display device 111 or send them to another information processing apparatus.

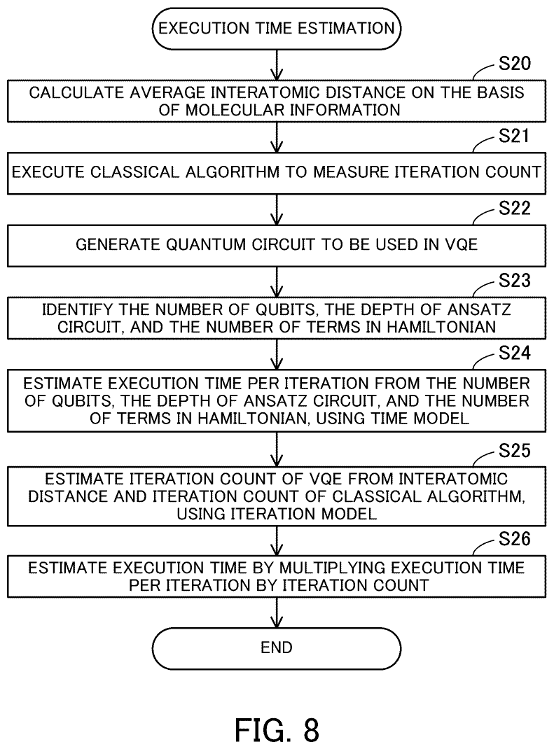

FIG. 8 is a flowchart illustrating an example procedure for execution time estimation. (S20) The execution time estimation unit 128 obtains molecular information specifying a molecule to be analyzed. The execution time estimation unit 128 calculates the average interatomic distance on the basis of the molecular information.

(S21) The CCSD execution unit 124 executes a classical algorithm (e.g., CCSD) on the basis of the molecular information and basis functions to measure the iteration count of the classical algorithm.

(S22) The VQE execution unit 125 performs the preprocessing of the VQE on the basis of the molecular information and basis functions to generate a quantum circuit to be used in the VQE.

(S23) The execution time estimation unit 128 identifies the number of qubits, the depth of an ansatz circuit, and the number of terms in a Hamiltonian from the preprocessing result obtained at step S22.

(S24) The execution time estimation unit 128 enters the number of qubits, the depth of the ansatz circuit, and the number of terms in the Hamiltonian, identified at step S23, into a trained time model to estimate an execution time per iteration.

(S25) The execution time estimation unit 128 enters the interatomic distance calculated at step S20 and the iteration count of the classical algorithm measured at step S21 into a trained iteration model to estimate the iteration count of the VQE.

(S26) The execution time estimation unit 128 estimates the execution time of the VQE by multiplying the execution time per iteration estimated at step S24 by the iteration count estimated at step S25. The execution time estimation unit 128 outputs the estimated execution time to the scheduler 129. The execution time estimation unit 128 may store the estimated execution time in a non-volatile storage device, display it on the display device 111, or send it to another information processing apparatus.

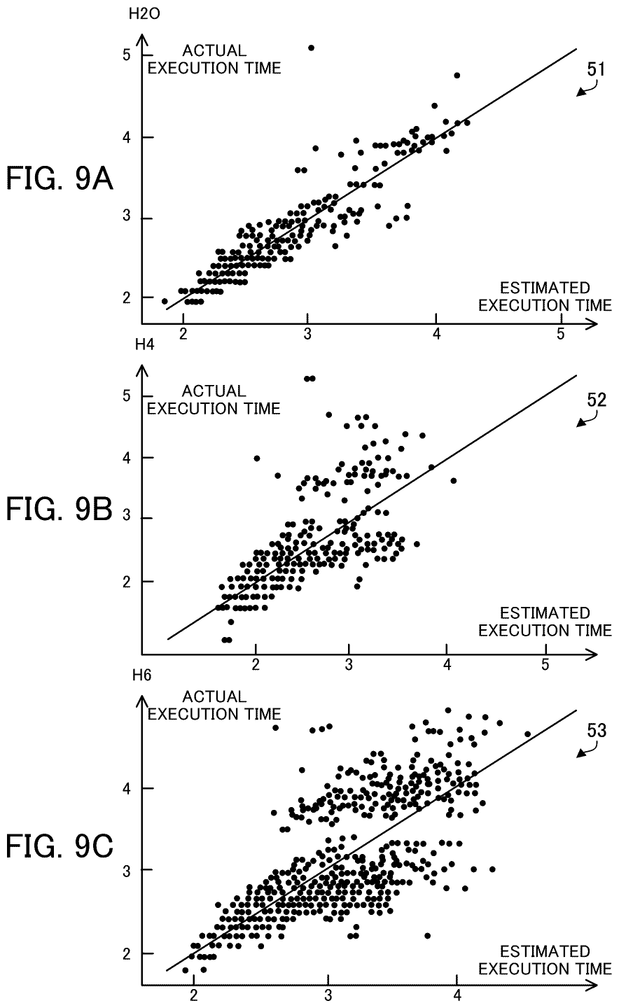

FIGS. 9A to 90 are graphs illustrating examples of execution time estimation accuracy. Graphs 51, 52, and 53 are scatter plots representing the relationship between the estimated execution time and the actual execution time with respect to the VQE. The estimated execution time here refers to an expected value of the execution time. In the graphs 51, 52, and 53, the horizontal axis represents the logarithm of the estimated execution time, whereas the vertical axis represents the logarithm of the actual execution time. In addition, each line segment indicates where the estimated execution time equals the actual execution time. That is to say, points closer to the line segment indicate samples with higher estimation accuracy.

The graph 51 represents simulation results regarding an H2O molecule, the graph 52 represents simulation results regarding an H4 molecule, and the graph 53 represents simulation results regarding an H6 molecule. The execution time of the VQE varies significantly depending on a molecular structure. Nevertheless, as represented in the graphs 51, 52, and 53, the information processing apparatus 100 is able to estimate the expected values close to the actual execution times.

In this connection, assuming the case of calculating the estimated execution times taking robustness into account or taking both robustness and uncertainty into account, the points in the graphs 51, 52, and 53 shift to the right. In this case, most points are positioned on the right side of the line segment, and only a few points are positioned on the left side thereof. This means that the risk of the execution time of the VQE exceeding the estimated value is sufficiently low, which enables the information processing apparatus 100 to perform efficient job scheduling.

As described above, the information processing apparatus 100 of the second embodiment estimates the execution time of the VQE before executing the VQE. This enables the information processing apparatus 100 to perform efficient job scheduling for quantum chemical calculation, on the basis of the estimated execution time. In addition, dividing the estimation of the execution time into the estimation of execution time per iteration and the estimation of iteration count, the apparatus 100 make these information processing estimations using different machine learning models. This approach uses feature values appropriate for calculating the respective estimated values, thereby improving the accuracy of estimating the execution time.

Furthermore, the information processing apparatus 100 executes a classical algorithm such as the CCSD to measure the iteration count of the classical algorithm, and uses this iteration count of the classical algorithm to estimate the iteration count of the VQE. The use of the iteration count of the classical algorithm that is correlated with the iteration count of the VQE improves the estimation accuracy compared to the case of estimating the iteration count of the VQE only from feature values of a quantum circuit. In addition, since the classical algorithm has a considerably shorter execution time than the VQE, the information processing apparatus 100 is able to quickly estimate the execution time of the VQE.

In addition, the information processing apparatus 100 further uses an interatomic distance in estimating the iteration count of the VQE. This enables the machine learning model to learn the tendency that the iteration count of the VQE increases as the interatomic distance becomes longer, thereby improving the accuracy of estimating the iteration count. In addition, considering an upside risk of the execution time of the VQE exceeding an expected value, caused due to factors such as randomness in the iteration count and insufficient training data, the information processing apparatus 100 calculates an estimated value that accounts for the risk. That is, the information processing apparatus 100 is able to reduce the risk of the execution time of the VQE exceeding the estimated value within an acceptable level, which makes it possible to achieve highly reliable job scheduling.

In one aspect, it is achieved to estimate the iteration count of a quantum chemical calculation algorithm.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A non-transitory computer-readable storage medium storing a computer program that causes a computer to perform a process comprising:
executing, among algorithms configured to obtain energy of a molecule through an iterative process and including a first algorithm and a second algorithm that uses quantum circuit data and is different from the first algorithm, the first algorithm based on molecular information specifying a molecule to be analyzed, to obtain a first iteration count of the first algorithm;
entering the first iteration count into a first machine learning model trained with an iteration count of the first algorithm as an explanatory variable and an iteration count of the second algorithm as a response variable; and
outputting an estimated value of a second iteration count of the second algorithm obtained from the first machine learning model, for execution of the second algorithm based on the molecular information.

2. The non-transitory computer-readable storage medium according to claim 1, wherein
the explanatory variable further includes a distance between a plurality of atoms in the molecule, and
the entering of the first iteration count further includes entering a first distance indicated in the molecular information.

3. The non-transitory computer-readable storage medium according to claim 1, wherein the process further includes
identifying a first feature value representing a feature of first quantum circuit data to be used in executing the second algorithm based on the molecular information,
entering the first feature value into a second machine learning model trained with a feature value of the quantum circuit data as an explanatory variable and a unit execution time per iteration of the iterative process of the second algorithm as a response variable, and
outputting an estimated value of a first unit execution time obtained from the second machine learning model, for the execution of the second algorithm based on the molecular information.

4. The non-transitory computer-readable storage medium according to claim 3, wherein the process further includes estimating an execution time, based on the estimated value of the second iteration count and the estimated value of the first unit execution time, for the execution of the second algorithm based on the molecular information.

5. The non-transitory computer-readable storage medium according to claim 4, wherein the process further includes scheduling a job that calculates energy of the molecule to be analyzed, based on the estimated execution time.

6. The non-transitory computer-readable storage medium according to claim 1, wherein
the first algorithm is a configuration interaction method or a coupled cluster method, and
the second algorithm is a variational quantum eigensolver.

7. An information processing apparatus comprising:
a memory configured to store a first machine learning model trained with an iteration count of a first algorithm as an explanatory variable and an iteration count of a second algorithm as a response variable, the first algorithm and the second algorithm being among algorithms configured to obtain energy of a molecule through an iterative process, the second algorithm using quantum circuit data; and
a processor coupled to the memory and the processor configured to
execute the first algorithm based on molecular information specifying a molecule to be analyzed, to obtain a first iteration count of the first algorithm,
enter the first iteration count into the first machine learning model, and
output an estimated value of a second iteration count of the second algorithm obtained from the first machine learning model, for execution of the second algorithm based on the molecular information.

8. A machine learning method comprising:
executing, by a processor, among algorithms configured to obtain energy of a molecule through an iterative process and including a first algorithm and a second algorithm that uses quantum circuit data and is different from the first algorithm, the first algorithm and the second algorithm based on molecular information specifying a sample molecule, to obtain a first iteration count of the first algorithm and a second iteration count of the second algorithm; and
training, by the processor, a first machine learning model using training data including the first iteration count and the second iteration count, with an iteration count of the first algorithm as an explanatory variable and an iteration count of the second algorithm as a response variable.
